(19) United States
(12) Patent Application Publication
(10) Pub. No.: US 2007/0282361 A1
(43) Pub. Date: Dec. 6, 2007

(54) Surgical Cutting Instrument

(75) Inventors: Orlando Da Rold, Solothurn (CH); Hassan Sadri, Riaz (CH)

Correspondence Address: Calvin G Covell;Tarolli Sundheim Covell & Tummino, 1300 East Ninth Street, Suite 1700, Cleveland, OH, 44114, US

(73) Assignee: ORLANDO DA ROLD, SOLOTHURN (CH)

(21) Appl. No.: 11/632,858

(22) PCT Filed: Jul. 20, 2005
(86) PCT No.: PCT/IB05/52425

§ 371 Date: Jun. 13, 2007

(30) Foreign Application Priority Data
Jul. 22, 2004 (CH) 01243/04

(51) Int. Cl. A61B 17/32

(52) U.S. Cl. 606/171

(57) ABSTRACT

A surgical cutting instrument is proposed which, by virtue of its geometric shape, can be used for all types of tissue. Surgical cutting instrument of great length, consisting of an outer tube (10) which, in the end area, is connected to an outer head part (11) which has a laterally and forwardly directed outer opening (12) with two outer cutting edges (121′, 121″), and of an inner tube (20) which, in the end area, is connected to an inner head part (21) which has, for example, three laterally and forwardly directed inner openings (22) within each case two inner cutting edges (221′, 221″). The inner tube (20) is mounted rotatably in the outer tube (10). Upon relative rotation of the inner tube (20) in the outer tube (10), the outer cutting edges (121′, 121″) and the inner cutting edges (221′, 221″) move past one another with slight clearance.

The present invention relates to a surgical cutting instrument according to the preamble of Patent Claim 1.

Surgical cutting instruments for use in closed or endoscopic surgery are known from EP 0 557 004, for example. They consist of two tubes which move one inside the other and on which the so-called cutting head is located in the end area at one end, and a coupling for the connection to the drive turbine is located at the other end. The end parts are open or closed to the front and also have forwardly directed lateral openings through which irrigation fluid and, during the intervention, detached tissue is carried off.

The tubes of such instruments are of small diameter and great length, so as to permit interventions on the body without causing large surgical wounds. The intervention is normally performed with three openings in the tissue, but in the area of the tissue closed to the outside. A camera for observing the operating site on screen is inserted through the first opening into the body, the second opening provides access for the surgical cutting instruments, and a nozzle is introduced through the third opening. This nozzle is used to deliver irrigation fluid to the operating site where the actual intervention is taking place. This irrigation fluid is again removed from the body, together with the detached tissue, through the tubular centre of the cutting tool.

To enlarge the operating site, for example in interventions on the hip joint, femur and pelvis, an invasive distractor is used, or simple stretching effected, to the extent permitted by the tendons, ligaments, nerves and muscles. The camera is used to view the operating site on TV monitors during the operation.

On the one hand, the operating site is kept clean by the irrigation fluid, and, on the other hand, the parts detached by the cutting instrument are suctioned off from the operating site together with the irrigation fluid. Instruments are also commercially available which have a camera through which the irrigation fluid at the same time is brought to the operating site. The combined device of camera with delivery of fluid makes it possible to work with just two intervention sites. In many cases, the combination of camera with fluid delivery is not possible for practical reasons, for example in cases when technical operating reasons mean that the camera should be moved to another location in order to gain a quite specific view of the operating site.

As has been described above, the surgical cutting instruments to which the invention pertains are designed as long tubes. The irrigation fluid, together with the detached parts, is suctioned off through the inner, rotating tube. Important problems to be solved when producing such surgical cutting instruments are the production technique, the material selection, and the economic aspects. Suitable sterilizable material must be processed such that, with economic production, it is possible to obtain a cutting instrument that is as precise as possible. Normally, such instruments are made of expensive metal alloys.

To be able to operate as efficiently as possible with the surgical cutting instrument, it should have sharp cutting edges. The precision with which the cutters of the inner tube and the cutters of the outer tube approximate to one another is, together with the sharp edges of the cutters, crucial in determining how easily tissue can be detached. The most demanding requirement placed on the surgical cutting instrument is that, in one and the same intervention, different types of tissue structures have to be cut and removed. It is well known that a cutting tool for brittle and hard material is not readily suitable for cutting elastic, fibrous or plastically soft materials. This, however, is exactly what the surgeon wants if he is to perform the operation efficiently and in a short time.

This requirement is met by EP 0 557 044, for example, to the extent that the cutting edges of the inner tube are provided with sawtooth-like cutters. This has the effect that elastic tissue and hard tissue can be “grabbed”. Here, however, the problem with this type of design of the cutting edge still remains. The tool grabs at tissue and has a tendency to catch in the tissue. If the cutting instrument catches, it either comes to a standstill through the response of an overload coupling, or it tears out too much of the tissue to be treated.

Conventional surgical cutting tools are rounded at the front end. This is based on the belief that a rounded instrument can be more easily introduced into the tissue. It is also based, however, on the more simple production technique when a closure piece is to be arranged on a long tube. Methods such as thermoforming or flanging, etc., can be used. All these surgical cutting tools present on the market are inexact from the mechanical point of view and from the point of view of tolerances. The production techniques do not allow for anything else. In addition, material that can be thermoformed must be soft material, it cannot be brittle or hard. The inventor knows of no tools of this type which would be hardened.

In surgery, experience shows that this can have the following disadvantageous effects listed below:
The soft material of the outer tube and of the inner tube can “erode”.
The material must be nickel-plated for the surgical cutting tools, which entails the risk of this layer crumbling off.
The methods of thermoforming or flanging never achieve a nicely defined surface, and no rounding can be exactly determined.
Non-circular, hard inner tubes which rotate in non-circular outer tubes made of soft material cause metal parts to abrade, which can remain in the tissue and cause problems.

The present invention now has the object of improving a surgical cutting instrument of the type mentioned at the outset in such a way that it easily satisfies the stringent conditions for in vivo use and provides the surgeon with a really sharp cutting instrument for all types of tissue and bones.

This object is achieved by a surgical cutting instrument having the features of Patent Claim 1. Further features according to the invention will become evident from the dependent claims, and their advantages are explained in the following description.

In the drawing:

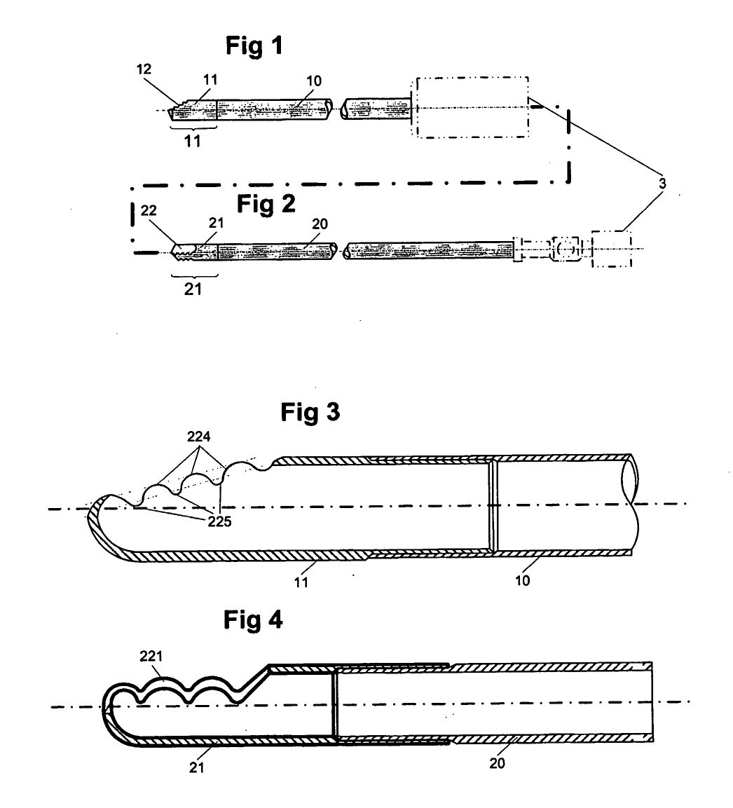

FIG. 1 shows a view of the outer tube with coupling element,

FIG. 2 shows a view of the inner tube with coupling element,

FIG. 3 shows a longitudinal section through the outer tube,

FIG. 4 shows a longitudinal section through the inner tube,

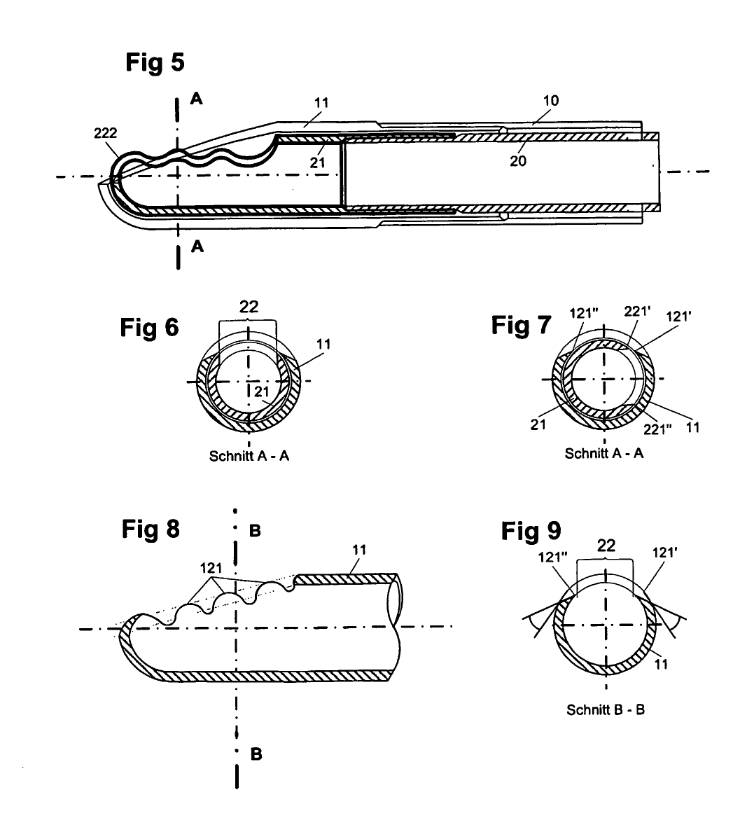

FIG. 5 shows a longitudinal section through assembled outer tube and inner tube,

FIG. 6 shows a cross section at location A-A through assembled outer and inner tubes,

FIG. 7 shows a cross section at location A-A through assembled outer and inner tubes,

FIG. 8 shows a longitudinal section through the outer head part,

FIG. 9 shows a cross section at location B-B through an outer head part,

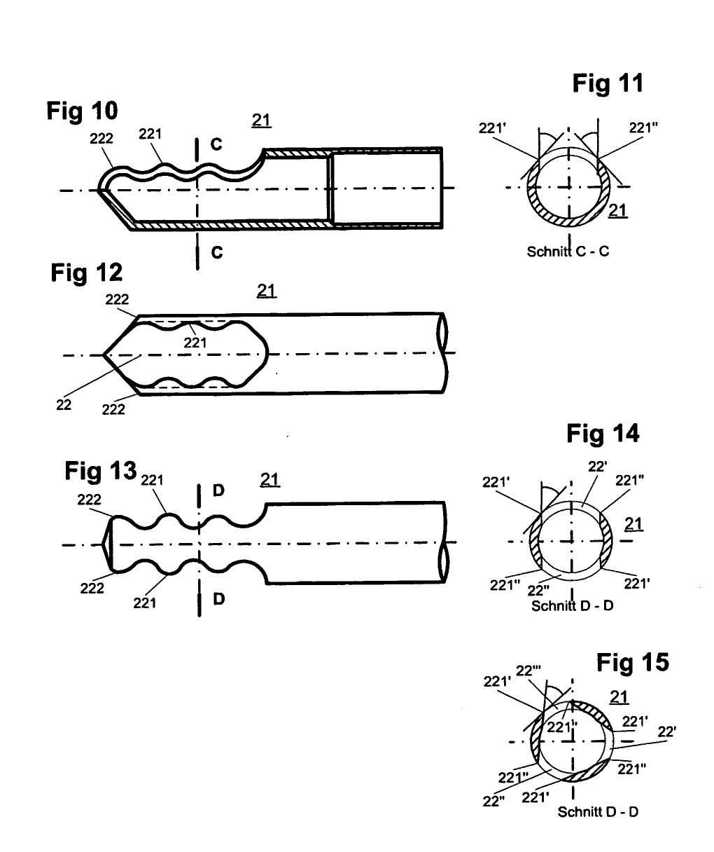

FIG. 10 shows a longitudinal section through the inner head part,

FIG. 11 shows a cross section at location C-C through an inner head part with one inner opening,

FIG. 12 shows a view of an inner head part with one or two inner openings,

FIG. 13 shows a view of an inner head part with one or two inner openings turned through 90°,

FIG. 14 shows a cross section at location D-D through an inner head part with two inner openings,

FIG. 15 shows a cross section at location D-D through an inner head part with three inner openings,

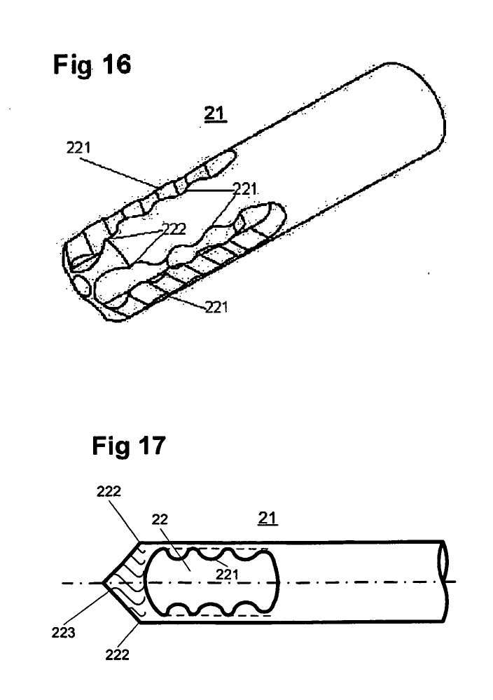

FIG. 16 shows a three-dimensional view of an inner head part with three inner openings,

FIG. 17 shows a view of an inner head part with one or two inner openings and a milling head at the tip.

The figures depict preferred illustrative embodiments which are explained in the description below.

A surgical cutting instrument according to the prior art of this invention consists of a fixed outer tube 10 (FIG. 1) and of an inner tube 20 (FIG. 2) rotating in the latter. The whole cutting instrument is connected to a drive mechanism provided for it, so that the inner tube 20 can in most cases be driven by a turbine drive and, at the same time, suction is guaranteed through the centre of the tubular instrument by means of vacuum. Such devices are used widely in arthroscopic treatment for small surgical interventions.

In known designs, outer tube 10 and outer head part 11 are made as one piece. This applies also to the inner tube 20 and inner head part 21. Since, in clinical use, such instruments must be kept absolutely clean and sterile, special alloys are used for their production. The demanding technique of welding such materials and material thicknesses is the reason why the tubes are usually produced in one piece. The inventor has now developed methods which permit secure welding between outer tube 10 and outer head part 11 (FIG. 3) and also between inner tube 20 and inner head part 21 (FIG. 4). In this way, other processing methods are possible for the head parts.

The cutting device according to the invention differs significantly from the commercially available products. The outer cutting edges 121′ and 121″ of the outer head part 11 form inwardly with respect to the tangent an acute angle α. The outer cutting edges 121′ and 121″ are thus situated directly on the internal diameter of the outer head part 11. To ensure that only a small cutting force is obtained for good cutting properties, the inner cutting edges 221′ and 221″ form outwardly with respect to the tangent an acute angle β, so that the inner cutting edges 221′ and 221″ are situated on the external diameter of the inner head part 21. If the inner head part 21 now turns in the clockwise direction (FIG. 7), the inner cutting edge 221′ moves very close past the outer cutting edge 121′. The chosen geometry of the shape of a toothing along the length of the cutting edges 121, 221 affords the effect that the cutting edges 121 and 221 have the tendency to come together during the cutting procedure. This once again gives an improved cutting property, because in this way practically no clearance is now present between the cutting edges 121 and 221. Since both cutting edges, both the outer cutting edge 121′ and also the inner cutting edge 221′, have a small cutting angle, any type of tissue or bone, whether soft, elastic, hard or fibrous, is cleanly cut off. No tissue is pinched off or even torn off. The same applies on reverse rotation between inner cutting edge 221″ and outer cutting edge 121″.

The cutting instrument can be used in both directions of rotation and oscillating with exactly the same cutting property and cutting quality. Cutting instruments of smaller diameter can thus be used. This is unique and in particular affords considerable advantages in confined operating conditions. An example of this is an operation performed on the meniscus. For interventions on joints, the extent of the distraction can thus be kept smaller, which reduces the risk of overextension of tendons, muscles and nerves.

The shape of this cutting instrument permits use in many areas even where milling tools would traditionally be used. The outer cutting edges 121 (FIG. 3) and the inner cutting edges 221 (FIG. 10) have an undulating configuration along their length. In this way there are practically no moments at which the whole cutter is simultaneously in use. This makes it possible to work efficiently and quickly with small drive forces. The wave shape of the teeth shown in FIG. 3, wide tooth 224 and narrow tooth gap 225, is typical for this surgical cutting instrument.

As has been described above, conventional cutting tools are rounded at the front end. As has been mentioned above, in the proposed surgical cutting instrument the outer head part 11 and the inner head part 21 can be connected, after production, to the outer tube 10 and the inner tube 20, respectively. This permits other shapes and the use of other production techniques for forming the two head parts 11, 21.

The inventor proposes in particular a conical closure of the surgical cutting blade. As is shown in FIG. 5, in this design of the outer head part 11 and of the inner head part 21, a cutting corner 222 is obtained which protrudes slightly from the cutting edge 121 of the outer head part 11. With this cutting corner 222, the surgeon is able to cut with great precision. A common method, for example, is to use the lateral cutting corner 222 to cut a pit-like recess in the flat tissue which is to be carried off. On the monitor, he can thus tell how much material he has cut from the surface around the furrow. This is a great help as reference line for estimating the amount of material removed during the operation. On the monitor, the relative amounts can be ascertained only with great experience and only inexactly. This form of the surgical cutting instrument thus permits more rapid working, without at the same time entailing a greater risk.

The greatest novelty, made possible by recent production techniques and the choice of the right materials, is the formation of the inner head part with one, two or three inner openings 22 (FIG. 12). The problem to be solved was the strength of the material of the inner head part. This material has to be very tough but at the same time weldable. A material having both properties was found. The great advantage of using two (FIGS. 13, 14) or even three (FIG. 15) inner openings 22 is appreciated when suctioning off irrigation fluid and tissue. The better the cutting performance and the greater the opening through which the detached material can be carried off, the cleaner is the operating site and, therefore, the quicker and more precise the surgeon's work.

The outer tube head part 11 remains the same in all configurations of the inner tube head parts 21. Each inner opening, whether one, two or three are present, has in each case two inner cutting edges 221′, 221″. These are also always of the same design, so that work can be carried out in both directions of rotation using the cutting instrument with one inner opening (FIGS. 10, 11, 12), with two inner openings (FIGS. 13, 14) or with three inner openings (FIG. 15).

In practical use, it is necessary also to fit a milling head 223 (FIG. 17). In order to exploit the advantages of the above-described surgical cutting tool 1 and still have a milling tool available, the conical closure of the inner head part 21 was designed as a milling head 223 on a surgical cutting tool 1. This arrangement has the advantage that the material milled off passes immediately to the inner opening 22 and can be suctioned off there. This milling head 223 is designed as a “cap” with a plurality of milling teeth. The largest possible number of milling teeth is desirable.

The design of the milling head 223 as a cap, together with the fact that the proposed surgical cutting tool works with extremely small clearances, has the advantage that no soft parts are wound round the milling head 223. This is one of the great problems that conventional milling tools often have in this application, especially if the outer head part 11 is designed so that the milling head 223 is absolutely free to the front. As is shown in FIG. 8 and FIG. 9, the outer tube head part 11 always remains the same, ever for use of this inner head part 21 equipped with milling head 233.

Trials clearly showed that a far greater cutting performance can be achieved at the same speed of rotation of the driven inner tube 20. It was also found that with the tools equipped according to the invention, in particular also with the inner head part 21 with milling head 223 (FIG. 17), much less force has to be applied than with conventional tools. Further advantages found in practical use are:
The proposed cutting instrument permits more rapid working and is therefore, not just for economy-related reasons, superior to the conventional tools.
The proposed cutting instrument can be used as a cutter (without milling head 223 on the inner head part 21) and as milling blade (with the milling head 223 on the inner head part 21). The areas of use overlap such that both tools can be used to work in areas which require a number of known tools and thus require tools to be changed during the operation.
The chosen shape of the cutting geometry reduces the risk of injury during introduction of the tool into the tissue.
The round teeth are much more robust than the pointed teeth of the known tools.
The conical design of the inner head part 21 allows same to be used as drill.
The production of the head parts (inner head part 21 and outer head part 11) separate from the tubes (inner tube 20 and outer tube 10) permits small and always identical tolerances, irrespective of the diameter of the tool.

1. Surgical cutting instrument of great length, consisting of an outer tube (10) which, in the end area (2), is connected to an outer head part (11) which has at least one laterally and forwardly directed outer opening (12) with at least two outer cutting edges (121′, 121″), and of an inner tube (20) which, in the end area (2), is connected to an inner head part (21) which has at least one laterally and forwardly directed inner opening (22) with at least two inner cutting edges (221′, 221″), the inner tube (20) being mounted rotatably in the outer tube (10), and, upon relative rotation of the inner tube (20) in the outer tube (10), the outer cutting edges (121′, 121″) and the inner cutting edges (221′, 221″) move past one another with slight clearance, wherein the outer cutting edges (121′, 121″) form inwardly with respect to the tangent an acute angle α of 10-90° and extend in an undulating configuration along the length, and the inner cutting edges (221′, 221″) form outwardly with respect to the tangent an acute angle β of 10° to 90° and extend in an undulating configuration along their length.

2. Cutting instrument according to claim 1, wherein the outer head part (11) and the inner head part (21) in the end area form a spherical shape for closure.

3. Cutting instrument according to claim 1, wherein the outer head part (11) and the inner head part (21) in the end area form a conical shape for closure.

4. Cutting instrument according to claim 1, wherein the inner head part (21) has two inner openings (22′ 22″) within each case two inner cutting edges (221′, 221″)

5. Cutting instrument according to claim 1, wherein the inner head part (21) has two inner openings (22′, 22″) within each case two inner cutting edges (221′, 221″).

6. Cutting instrument according to claim 1, wherein the inner head part (21) has three inner openings 22′, 22″, 22′″) within each case two inner cutting edges (221′, 221″).

7. Cutting instrument according to claim 1, wherein the inner head part (21) has three inner openings (22′, 22″, 22′″) within each case two inner cutting edges (221′, 221″).

8. Cutting instrument according to claim 1, wherein the inner head part (21) has a milling head (223) at the end.

9. Cutting instrument according to claim 1, wherein the upright teeth (224) are at least twice as wide as the tooth gaps (225).
